(19) United States
(12) Patent Application Publication
(10) Pub. No.: US 2004/0152701 A1
(43) Pub. Date: Aug. 5, 2004

(54) Novel anhydrous crystalline form of Levofloxacin and process for preparation there of

(75) Inventors: Manne Satyanarayana Reddy, Hyderabad (IN); Sajja Eswaraiah, Hyderabad (IN); Koppera Ravinder Reddy, Hyderabad (IN); Maram Reddy Sahadeva Reddy, Hyderabad (IN); Pitta Jaya Prakash, Hyderabad (IN)

Correspondence Address: Ladas & Parry, 26 West 61 Street, New York, NY, 10023, US

(73) Assignees: DR. REDDY'S LABORATORIES LIMITED; DR. REDDY'S LABORATORIES, INC.

(21) Appl. No.: 10/716,207

(22) Filed: Nov. 18, 2003

(30) Foreign Application Priority Data
Dec. 2, 2002 (IN) 898/MAS/2002

(51) Int. Cl. A61K 31/58; C07D 491/02

(52) U.S. Cl. 514/230.5; 544/105

(57) ABSTRACT

The present invention is directed to novel anhydrous crystalline form of Levofloxacin is depicted as Formula (I), further more its process for preparation thereof. The process for the preparation of novel anhydrous crystalline form of Levofloxacin comprises the condensation of N-Methyl piperazine with S(−)-9,10-difluoro-7-oxo 2,3-dihydro 7H-pyrido[1,2,3-DE] [1,4] Benzoxazine-6-carboxylic acid in Acetonitrile followed by distillation of solvent to afford the residue, the resultant residue is refluxed with toluene and the solid is filtered at room temperature to afford the Levofloxacin. Thus resulted Levofloxacin is further refluxed in Acetonitrile and filtered the Novel anhydrous crystalline form of Levofloxacin as undissolved material. The anhydrous crystalline form of Levofloxacin is characterized by X-ray diffractogram, Differential Scanning Calorimetry thermogram and Infrared Spectra.

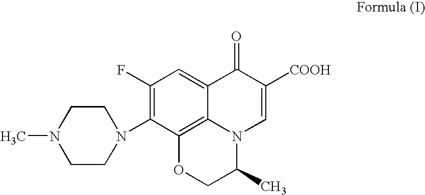

### FIELD OF THE INVENTION

The present invention relates to the crystalline form of S (−)-9-fluoro-2,3-dihydro-3-methyl-10-(4-methyl-1-piperazinyl)-7-oxo-7H-pyrido[1,2,3-de]-1,4-benzoxazine-6-carboxylic acid (Levofloxacin). More specifically, the present invention relates to novel anhydrous crystalline form of Levofloxacin and process for preparation there of. Levofloxacin can be depicted as Formula (1).

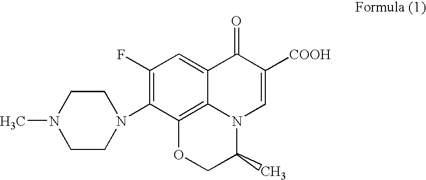

### BACKGROUND OF THE INVENTION

Levofloxacin is an antimicrobial agent. It is a quinolone antibiotic used to treat lung, skin and urinary tract infections caused by bacteria.

U.S. Pat. No. 5,053,407 specifically claimed Levofloxacin along with its process and method of treatment. The process for the preparation of Levofloxacin comprises the condensation of N-Methyl piperazine with of S (−)-9,10-difluoro-7-oxo 2,3-dihydro 7H-pyrido [1,2,3-DE] [1,4] Benzoxazine-6-carboxylic acid in Dimethyl sulfoxide followed by the isolation of the pure Levofloxacin by chromatographic techniques.

U.S. Pat. No. 5,545,737 discloses the process for the preparation of Monohydrate and Hemihydrate of Levofloxacin. The process for the preparation of hemihydrate comprises treating crude Levofloxacin in an aqueous ethanol with relatively low water content ranging from 2 to 10%.

Further the process for the preparation of monohydrate comprises treating Levofloxacin hemihydrate slurry in water or an aqueous solvent with specific water content.

The said patent also disclosed the anhydrous form of Levofloxacin in general description and doesn't described the analytical data for anhydrous form.

The present invention relates to the novel anhydrous crystalline form of Levofloxacin and the present invention also relates to the process for the preparation of novel anhydrous crystalline form of Levofloxacin.

None of the prior art references related to Levofloxacin were described the crystalline structure for hydrate and anhydrous forms of Levofloxacin. The novel anhydrous form of Levofloxacin of the present invention is characterized by X-ray diffractogram and found that the pattern is different from any of the other hydrate forms of Levofloxacin.

The anhydrous form of Levofloxacin obtained in the present invention is having moisture content less than 0.5% by KF method in known perse; hence the present inventive substance can be referred as anhydrous form of Levofloxacin.

The present inventive substance is having a novel crystalline form, which has been characterized by its X-ray diffractogram, Differential Scanning Colorimetric thermogram and Infra red spectrum.

In general, the anhydrous crystalline forms of an active ingredient may have some advantageous over hydrated forms. The present inventive compound is free flowing and non-solvated crystalline solid; hence, the novel anhydrous crystalline form of Levofloxacin may be well suitable for pharmaceutical formulations.

Another aspect of the present invention is to prepare a novel anhydrous crystalline form of Levofloxacin in a simple, eco-friendly and commercially viable process.

### SUMMARY OF THE INVENTION

The present invention is directed to novel anhydrous crystalline form of Levofloxacin, further more its process for preparation thereof. The process for the preparation of novel anhydrous crystalline form of Levofloxacin comprises the condensation of N-Methyl piperazine with S (−)-9,10-difluoro-7-oxo 2,3-dihydro 7H-pyrido [1,2,3-DE] [1,4] Benzoxazine-6-carboxylic acid in Acetonitrile followed by distillation of solvent to afford the residue, the resultant residue is refluxed with toluene and the solid is filtered at room temperature to afford the Levofloxacin. Thus resulted Levofloxacin is further refluxed in Acetonitrile and filtered the Novel anhydrous crystalline form of Levofloxacin as undissolved material. The anhydrous crystalline form of Levofloxacin is characterized by X-ray diffractogram, Differential Scanning Calorimetry thermogram and Infrared Spectra.

### BRIEF DESCRIPTTION OF THE ACCOMPANYING DRAWINGS

FIG. 1 is a diagram showing the results of X-ray diffraction of the inventive substance.

FIG. 2 is a diagram showing the results of Infra Red Spectrum of the inventive substance.

### DETAILED DESCRIPTION OF THE INVENTION

The present invention provides the novel anhydrous crystalline form of Levofloxacin and a process for the preparation thereof.

The crystalline nature of novel anhydrous form of Levofloxacin of present invention is characterized by its X-ray diffractogram, Differential Scanning colorimetry thermogram and IR spectra.

The anhydrous nature of the inventive substance was characterized by its thermo gravimetric analysis, and the anhydrous nature of the compound was also confirmed by calculating the water content present in the compound by Karl Fischer (KF) method.

The report of thermo gravimetric analysis shows a total weight loss of 0.1% at a temperature range of 25-300° C. The result indicates the anhydrous nature of the inventive substance obtained as per Example (1) of experimental section.

The present inventive substance is having a moisture content of 0.34% by KF method, which confirms the anhydrous nature of the compound.

The X-ray powder diffraction pattern of anhydrous crystalline form of Levofloxacin is measured on a Bruker Axs, D8 Advance Powder X-ray Diffractometer with Cu K alpha-1 Radiation source.

The significant characteristic peaks identified in X-ray powder diffractogram are shown in the Table-1. The X-ray powder diffraction pattern is expressed in terms of the 2 theta (degrees), and percentage of intensity (in %).

TABLE-1

| | Intensity |
|---|---|
| 2 theta (°) | (I/Iₒ) |
| 12.471 | 100 |
| 26.197 | 58.6 |
| 17.328 | 17.7 |
| 13.777 | 27.1 |
| 21.088 | 24.7 |
| 10.12 | 19.1 |
| 18.958 | 17.7 |
| 9.433 | 13.6 |
| 26.724 | 13.3 |
| 15.678 | 6.7 |
| 15.11 | 5.7 |
| 23.048 | 5.2 |
| 24.419 | 5.2 |
| 23.683 | 5.0 |
| 6.281 | 4.3 |
| 27.781 | 3.6 |
| 33.121 | 3.6 |
| 35.226 | 3.5 |
| 37.536 | 2.5 |
| 28.671 | 2.2 |
| 29.929 | 2.2 |
| 39.07 | 2.2 |
| 16.137 | 2.1 |
| 20.341 | 1.8 |
| 42.077 | 1.8 |
| 21.767 | 1.7 |
| 25.051 | 1.5 |
| 19.782 | 1.2 |
| 27.188 | 0.9 |

The X-ray diffractogram of the novel anhydrous crystalline form of Levofloxacin is substantially as depicted in FIG. (1).

The present invention provides the Differential Scanning Calorimetry thermogram of anhydrous crystalline form of Levofloxacin. The Differential Scanning Calorimetry thermogram exhibits a significant exo peak at 272° C.

The present invention further provides the Infrared spectral data for anhydrous crystalline form of Levofloxacin, which was measured by KBr-transmission method with identified significant peaks at about 460.2, 481.0, 541.9, 560.9, 581.2, 654.6, 671.5, 745.1, 803.1, 839.2, 872.9, 902.1, 937.8, 949.0, 979.6, 1021.6, 1049.6, 1084.7, 1193.9, 1249.5, 1293.5, 1305.0, 1342.1, 1397.1, 1450.3, 1521.6, 1546.9, 1621.3, 1726.2, 2782.2, 3020.0 and 3041.7 cm−¹.

The present invention provides the IR spectrum of anhydrous crystalline form of Levofloxacin and substantially as depicted in FIG. (2).

Another embodiment of the present invention is to provide the process for the preparation of novel anhydrous crystalline form of Levofloxacin, which comprises;

i) refluxing N-Methyl piperazine with S (−)-9,10-difluoro-7-oxo 2,3-dihydro 7H-pyrido [1,2,3-DE] [1,4] Benzoxazine-6-carboxylic acid in nitrile solvents such as acetonitrile or propionitrile, preferably acetonitrile till the reaction substantially completes;

ii) distilling off the solvent from the reaction solution obtained in step (i);

iii) refluxing the residue obtained in step (ii) in aromatic hydrocarbon solvent comprising of benzene, toluene, xylene or ethyl benzene, preferably toluene for 1 to 10 hours;

iv) cooling the reaction mass obtained in step (iii) to 0-25° C. to obtain solid mass;

v) filtering the solid mass and drying at a temperature of 30-100° C., preferably at 40-50° C. to get the crude compound;

vi) refluxing the crude compound obtained in step (v) in nitrile solvents comprising of acetonitrile or propionitrile, preferably acetonitrile;

vii) filtering the undissolved material obtained in step (vi) and further washing with nitrile solvent as described in step (i);

viii) drying the filtered undissolved material of step (vii) at 30-100° C., preferably at 40-50° C. to afford the novel anhydrous crystalline form of Levofloxacin.

Thus, the present invention is directed to a novel anhydrous crystalline form of Levofloxacin and the compound obtained in the above process is free flowing and non-solvated, which renders it well suited for pharmaceutical formulations.

The carboxylic acid derivative, which is the starting material of the present invention is prepared as per the process disclosed in prior references.

The present invention is illustrated by the following examples, which are not intended to limit the effective scope of the claims.

Preparation of Novel Anhydrous Crystalline Form of Levofloxacin:

### EXAMPLE-1

S (−)-9,10-difluoro-7-oxo 2,3-dihydro 7H-pyrido [1,2,3-DE] [1,4] Benzoxazine-6-carboxylic acid (75 grams), N-Methyl piperazine (67 grams) were suspended in Acetonitrile (525 ml) and heated to the reflux temperature, further stirred till the reaction substantially completes. The solvent was distilled off completely from the reaction mixture to get the residual mass. Thus resulted residual mass was refluxed with toluene (75 ml) for about 5-6 hours then cooled to a temperature of 0-5° C. and stirred for 2-4 hours. The obtained solid mass was filtered and washed with toluene (75 ml) and dried at a temperature of 40-50° C. to a constant weight (45 grams). The dried compound was further refluxed with acetonitrile (675 ml) for 30-60 minutes and filtered the undissolved compound and washed with acetonitrile (45 ml). Thus obtained undissolved compound was dried at a temperature of 40-50° C. to a constant weight to afford desired Novel anhydrous crystalline form of Levofloxacin.

(Weight: 24 grams, M.C. by KF is 0.34%).

### DETAILED DESCRIPTION OF THE ACCOMPANYING DRAWINGS

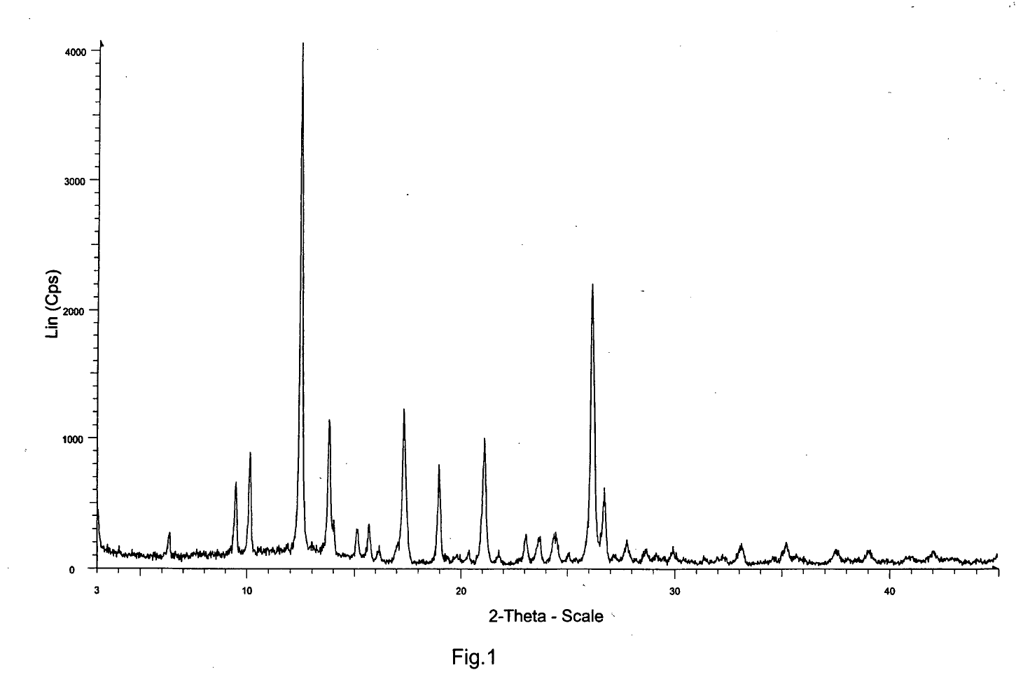

FIG. 1 is characteristic X-ray powder diffraction pattern of the novel anhydrous crystalline form of Levofloxacin.

Vertical axis: Intensity (CPS); Horizontal axis: 2 Theta (degrees).

The significant 2 theta values (in degrees) obtained are 6.281, 9.433, 10.12, 12.471, 13.777, 15.11, 15.678, 16.137, 17.328, 18.958, 19.782, 20.341, 21.088, 21.767, 23.048, 23.683, 24.419, 25.051, 26.197, 26.724, 27.188, 27.781, 28.671, 29.929, 33.121, 35.226, 37.536, 39.07 and 42.077 degrees two theta.

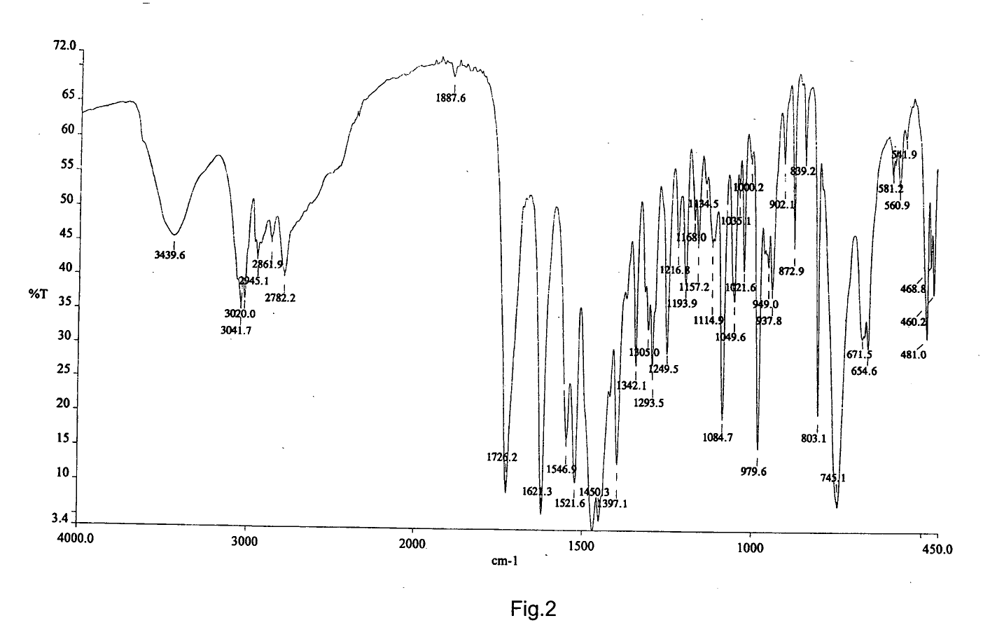

FIG. 2 is characteristic Infra Red spectrum of anhydrous crystalline form of Levofloxacin with identified significant peaks at about 460.2, 481.0, 541.9, 560.9, 581.2, 654.6, 671.5, 745.1, 803.1, 839.2, 872.9, 902.1, 937.8, 949.0, 979.6, 1021.6, 1049.6, 1084.7, 1193.9, 1249.5, 1293.5, 1305.0, 1342.1, 1397.1, 1450.3, 1521.6, 1546.9, 1621.3, 1726.2, 2782.2, 3020.0 and 3041.7 cm−¹.

Vertical axis: Wave length (in Cm⁻¹); Horizontal axis: Transmission (in %).

We claim:

1. A novel anhydrous crystalline form of S (−)-9-fluoro-2,3-dihydro-3-methyl-10-(4-methyl-1-piperazinyl)-7-oxo-7H-pyrido[1,2,3-de]-1,4-benzoxazine-6-carboxylic acid (Anhydrous Levofloxacin)

2. The anhydrous crystalline form of Levofloxacin of claim 1 has X-ray powder diffraction pattern with peaks around 6.281, 9.433, 10.12, 12.471, 13.777, 15.11, 15.678, 16.137, 17.328, 18.958, 19.782, 20.341, 21.088, 21.767, 23.048, 23.683, 24.419, 25.051, 26.197, 26.724, 27.188, 27.781, 28.671, 29.929, 33.121, 35.226, 37.536, 39.07 and 42.077 degrees two theta.

3. The anhydrous crystalline form of Levofloxacin of claim 2 having an X-ray powder diffraction pattern substantially as depicted in FIG. (1).

4. The anhydrous crystalline form of Levofloxacin of claim 1 having an identified significant characteristic peaks at about 460.2, 481.0, 541.9, 560.9, 581.2, 654.6, 671.5, 745.1, 803.1, 839.2, 872.9, 902.1, 937.8, 949.0, 979.6, 1021.6, 1049.6, 1084.7, 1193.9, 1249.5, 1293.5, 1305.0, 1342.1, 1397.1, 1450.3, 1521.6, 1546.9, 1621.3, 1726.2, 2782.2, 3020.0 and 3041.7 cm−¹ in the Infra red Spectrum.

5. The anhydrous crystalline form of Levofloxacin of claim 4 having an Infra red Spectrum substantially as depicted in FIG. (2).

6. A process for preparing the novel anhydrous crystalline form of S(−)-9-fluoro-2,3-dihydro-3-methyl-10-(4-methyl-1-piperazinyl)-7-oxo-7H-pyrido[1,2,3-de]-1,4-benzoxazine-6-carboxylic acid (Anhydrous Levofloxacin), which comprises;
i) refluxing N-Methyl piperazine with S(−)-9,10-difluoro-7-oxo2,3-dihydro7H-pyrido[1,2,3-DE] [1,4] Benzoxazine-6-carboxylic acid in nitrile solvents such as acetonitrile or propionitrile, preferably acetonitrile till the reaction substantially completes;
ii) distilling off the solvent from the reaction solution obtained in step (i);
iii) refluxing the residue obtained in step (ii) in aromatic hydrocarbon solvent comprising of benzene, toluene, xylene or ethyl benzene, preferably toluene for 1 to 10 hours;
iv) cooling the reaction mass obtained in step (iii) to 0-25° C. to obtain solid mass;
v) filtering the solid mass and drying at a temperature of 30-100° C., preferably at 40-50° C. to get the crude compound;
vi) refluxing the crude compound obtained in step (v) in nitrile solvents comprising of acetonitrile or propionitrile, preferably acetonitrile;
vii) filtering the undissolved material obtained in step (vi) and further washing with nitrile solvent as described in step (i);
viii) drying the filtered undissolved material of step (vii) at 30-100° C., preferably at 40-50° C. to afford the novel anhydrous crystalline form of Levofloxacin.

7. The process according to claim 6 of step (i), (vi) and (vii) where in the nitrile solvent is acetonitrile.

8. The process according to claim 6 of step (iii) where in the aromatic hydrocarbon solvent is toluene.

9. The process for the preparation of novel anhydrous crystalline form of Levofloxacin is substantially as herein described and exemplified.
